(19) United States
(12) Patent Application Publication
(10) Pub. No.: US 2003/0157172 A1
(43) Pub. Date: Aug. 21, 2003

(54) Pharmaceutical suspension for oral administration

(76) Inventors: Guang Wei Lu, Ann Arbor, MI (US); Po-Chang Chiang, Ballwin, MO (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/314,684

(22) Filed: Dec. 9, 2002

Related U.S. Application Data
(60) Provisional application No. 60/342,599, filed on Dec. 20, 2001.

(51) Int. Cl. A61K 9/26; A61K 31/075

(52) U.S. Cl. 424/469

(57) ABSTRACT

A pharmaceutical composition suitable for oral administration is provided, the composition comprising an aqueous medium having suspended therein a solid substance of low water solubility in particulate form, and further comprising a suspending agent and at least one pharmaceutically acceptable water-soluble or swellable nonsurfactant polymer, the total amount of all such polymers present being ineffective to (a) increase viscosity of the composition to a degree that significantly impairs pourability, or (b) significantly increase rate of sedimentation or phase separation, of the composition. The composition has improved mouth feel.

This application claims the priority of U.S. provisional application Serial No. 60/342,599 filed on Dec. 20, 2001.

### FIELD OF THE INVENTION

The present invention relates to suspension formulations suitable for oral administration of substances including drugs, other bioactive substances and placebo solids. In particular it relates to suspension formulations of such substances having low solubility in water.

### BACKGROUND OF THE INVENTION

The most common dosage forms used for oral administration of drugs are tablets and capsules. However, for some drugs, some indications and some patient populations, tablets and capsules are less suitable. For example, administration of large doses of a drug in the form of tablets or capsules is often very inconvenient. Either a large number of tablets or capsules have to be administered, or individual tablets or capsules have to be large, and therefore difficult to swallow. This can result in impaired patient compliance. Certain patient groups, e.g., young children and the elderly, have particular difficulty in swallowing whole tablets or capsules, even if these are of moderate size.

Hence, in some instances drugs are formulated as liquid dosage forms for oral administration, for example as solutions, suspensions, elixirs and syrups. Such dosage forms are known to permit ease of administration and to increase compliance for patients who have difficulty in swallowing solid dosage forms. A yet further advantage of liquid dosage forms is that metering of dosages is continuously variable, providing infinite dose flexibility. The benefits of ease of swallowing and dose flexibility are particularly advantageous for infants, children and the elderly. An additional advantage is that a drug formulated in a liquid dosage form is often more rapidly absorbed after oral administration than if formulated in tablets or capsules.

The simplest liquid drug formulation is a solution of the drug. However, if solubility of the drug in a pharmaceutically acceptable carrier liquid, especially water, is not sufficient to enable formulation as a solution, a suspension of particles of the drug in such a carrier liquid can be an option. A suspension can also have advantages over a solution, for example in providing greater chemical stability of the drug or in overcoming an unpleasant taste.

A placebo suspension is often required for clinical or other trials, for example in a double blind clinical trial of a suspension formulation of a drug. The absence of drug particles in a placebo can significantly change appearance and mouth feel of the suspension by comparison with a suspension containing the drug, thereby reducing the usefulness of the drug-free suspension as a placebo since a clinician and/or a trial subject is readily able to tell the active from the placebo. A placebo suspension is required to be as nearly identical as possible to the drug suspension in appearance, smell, taste, mouth feel, viscosity and sedimentation rate.

Excipients are often added to an oral suspension of drug particles in order to make it more palatable. Examples of such excipients are sugars, artificial sweeteners and flavoring agents. In some cases, the drug particles are coated in order to mask an unpleasant taste. Although the taste of a suspension can be significantly improved by such means, a gritty mouth feel caused by the drug particles often remains.

Decreasing particle size of the drug and increasing viscosity of the suspension are known approaches to reducing such a gritty mouth feel. However, disadvantages are associated with both these approaches. Particle size reduction is a costly process requiring much energy. A high viscosity makes the suspension difficult to pour, creates problems in accurate dosage metering, and, while overcoming grittiness, can produce an unpleasant slimy feel in the mouth. Other approaches to improving mouth feel are to make particle shape more regular or to decrease surface roughness of the drug particles. However, these characteristics are often difficult to control.

Suspensions for oral administration can be either flocculated or deflocculated. The term “flocculated” herein refers to suspended solid drug and/or excipient particles that tend to coagulate and/or agglomerate together and settle to form loosely packed aggregates or flocs. A “flocculated suspension” herein is a suspension wherein at least a portion of all solid particles are flocculated particles. The term “substantially deflocculated” herein refers to suspended solid drug and/or excipient particles that do not coagulate and/or agglomerate together to form flocs. A “deflocculated suspension” herein is a suspension wherein substantially no solid particles are visibly flocculated.

Grittiness of mouth feel is typically a greater problem with flocculated suspensions. Where the flocculated particles are drug particles, preparation of a placebo suspension can result in a liquid having a very different mouth feel from the drug suspension. In this situation a clinician and/or a trial subject is readily able to tell the active from the placebo, reducing or destroying the value of a clinical trial using such a placebo suspension.

U.S. Pat. No. 5,147,655 to Ibsen, incorporated herein by reference, discloses combining particles containing an active substance with one or more gelling or swelling agents. These particles are adapted to be dispersed in an aqueous carrier, i.e., reconstituted, substantially immediately prior to administration. The swelling or gelling agents are said to be capable of forming a viscous medium around the particles in the aqueous carrier, hence masking gritty mouth feel and preventing adherence to oral mucosa.

This approach does not always work for a flocculated suspension because such gelling or swelling agents can significantly change viscosity of the suspension and surface properties ofthe particles, and thereby affect aggregation and sedimentation, which can be a problem especially in development of a placebo suspension. Addition of gelling or swelling agents as proposed in above-referenced U.S. Pat. No. 5,147,655 also tends to increase difficulty in pouring and/or metering the suspension, for example as a result of increase in viscosity.

U.S. Pat. No. 3,326,755 to Sheth, incorporated herein by reference, teaches use of alginic acid or alginates to reduce grittiness of liquid antacid compositions.

Suspensions for oral administration of nonsteroidal anti-inflammatory drugs (NSAIDs) in the form of nanoparticles, and comprising surface modifiers such as polymers in an amount of 0.1% to 90% by weight of dry particles, are disclosed in the patents cited below, all incorporated herein by reference.

U.S. Pat. No. 5,552,160 to Liversidge et al.

U.S. Pat. No. 5,591,456 to Franson & Snyder.

U.S. Pat. No. 5,718,919 to Ruddy & Roberts.

There remains a need in the art for orally deliverable drug suspensions, more particularly flocculated drug suspensions, having improved mouth feel, yet retaining low viscosity to avoid difficulty in pouring and/or a slimy mouth feel. There also remains a need in the art for placebo suspensions that mimic organoleptic properties of drug suspensions, in particular placebo suspensions that mimic mouth feel of drug suspensions.

### SUMMARY OF THE INVENTION

There is now provided a pharmaceutical composition suitable for oral administration, the composition comprising an aqueous medium having suspended therein a solid substance of low water solubility in particulate form, and further comprising a suspending agent and at least one pharmaceutically acceptable water-soluble or swellable nonsurfactant polymer, the total amount of all such polymers present being ineffective to (a) increase viscosity of the composition to a degree that significantly impairs pourability, or (b) significantly increase rate of sedimentation or phase separation, of the composition.

In particular, there is provided a pharmaceutical composition suitable for oral administration, the composition comprising an aqueous medium having suspended therein a solid substance of low water solubility in particulate form, and further comprising a suspending agent and at least one pharmaceutically acceptable water-soluble or swellable nonsurfactant polymer, the total amount of all such polymers present being less than 1%, preferably not greater than about 0.5%, by weight of the composition.

In one embodiment the solid substance in suspension is a drug substance in a therapeutically or prophylactically useful amount.

In another embodiment the solid substance in suspension is a particulate excipient and the composition is useful as a placebo suspension.

It has surprisingly been found that amounts of a water-soluble or swellable nonsurfactant polymer less than 1%, preferably not greater than about 0.5%, by weight, which are too low to increase viscosity of the suspension to a degree that significantly impairs pourability, nonetheless can significantly enhance mouth feel of the suspension. In previously described oral suspensions of drugs containing a water-soluble or swellable polymer, the amount of such polymer is relatively high, typically at least 1% by weight of the suspension, and is effective to substantially increase viscosity of the suspension.

The amount of polymer herein is expressed as percentage of total weight of the composition and not to percentage of weight of solid particles, as in some of the art referenced above.

Advantages of compositions of the invention include, without limitation, improved mouth feel, in particular reduced grittiness of mouth feel, compared to suspensions lacking a polymer as provided herein, improved pourability compared to suspensions having a greater amount of polymer than specified herein, and less slimy mouth feel than suspensions having greater amount of polymer than specified herein. Placebo suspensions of the invention have advantages including enhanced similarity to drug suspensions.

There is also provided a process for preparing a pharmaceutical composition of the invention, comprising a step of dispersing a solid particulate substance of low water solubility in an aqueous liquid to form a dispersion, a step of adding at least one water-soluble or swellable nonsurfactant polymer to the aqueous liquid, and a step of adding a suspending agent to the dispersion with mixing, to form a suspension, wherein the suspending agent is added after addition of the polymer. It has been found that this order of addition of the polymer and suspending agent is important to providing a suspension having acceptable viscosity, sedimentation rate and physical stability. The total amount of such polymer added is ineffective to (a) increase viscosity of the composition to a degree that significantly impairs pourability, or (b) significantly increase rate of sedimentation or phase separation, of the composition. Alternatively, the total amount of such polymer added is not more than about 0.5% by weight of the composition.

Also provided is a method of diagnosis, treatment or prevention of a disease or other adverse health condition in a subject, the method comprising orally administering a drug-containing suspension composition of the invention to the subject.

Also provided is a method of conducting a clinical trial of a suspension formulation of a drug, the method comprising use as a comparator of a placebo suspension of the invention.

### BRIEF DESCRIPTION OF THE DRAWING

FIG. 1 is a graph showing results of a study described in Example 4 to determine effect of increasing levels of particulates on the concentration of unbound povidone in a placebo suspension.

### DETAILED DESCRIPTION OF THE INVENTION

Novel pharmaceutical drug-containing compositions of the invention comprise one or more orally deliverable dose units. The term “orally deliverable” herein means suitable for oral administration. Absorption of a drug contained in a composition of the invention can occur in any part or parts of the gastrointestinal tract including the mouth, esophagus, stomach, duodenum, jejunum, ileum and colon. The term “dose unit” herein means a portion of a pharmaceutical composition that contains an amount of a drug suitable for a single oral administration to provide a therapeutic, prophylactic or diagnostic effect. The term “dose unit” herein does not carry an implication that individual dose units are necessarily discrete. Thus a suspension composition of the invention can be packaged in a single multi-dose container or in multiple single-dose containers. Typically one dose unit, or a small plurality (up to about 4) of dose units, provides a sufficient amount of the drug to result in the desired effect.

A drug-containing suspension of the invention comprises one or more drugs of low water solubility, whereof at least one is present in a particulate form. Although it is preferred that a substantial fraction of the drug is present in particulate form, another fraction ofthe drug can optionally be present in solution in the aqueous carrier liquid. Preferably at least about 50%, more preferably at least about 90%, e.g., at least about 98%, of the drug is present in particulate form.

“Particulate form” herein is defined as not being molecularly dissolved in a solvent, solubilized by means of a solubilizing agent or emulsified by means of an emulsifying agent, but instead being present as solid particles. Preferably the solid particles have a particle size of about 0.1 μm to about 1 mm, more preferably about 0.5 μm to about 500 μm, for example about 1 μm to about 50 μm. The term “particle size” herein refers to volume median diameter, as measured by any suitable technique, preferably using a laser diffraction instrument (e.g., Sympatec Helos). It should be noted that for particles having an irregular shape, such as acicular particles, the volume median diameter as measured by laser diffraction will be significantly smaller than the average of the longest dimension of the particle population.

Each dose unit or small plurality of dose units in a drug-containing composition of the invention comprises, in a therapeutically, prophylactically and/or diagnostically effective total amount, a drug of low water solubility. A drug or excipient of “low water solubility” herein refers to a compound having solubility in water, measured at 37° C., not greater than about 10 mg/ml, and preferably not greater than about 1 mg/ml. It is contemplated that compositions of the invention are especially advantageous for drugs or excipients having solubility in water, measured at 37° C., not greater than about 0.1 mg/ml.

Solubility in water for many pharmaceutically useful compounds can be readily determined from standard pharmaceutical reference books, for example The Merck Index, 11th ed., 1989 (published by Merck & Co., Inc., Rahway, N.J.); the United States Pharmacopoeia, 24th ed. (USP 24), 2000; The Extra Pharmacopoeia, 29th ed., 1989 (published by Pharmaceutical Press, London); and the Physicians Desk Reference (PDR), 2001 ed. (published by Medical Economics Co., Montvale, N.J.).

For example, individual compounds of low solubility as defmed herein include compounds categorized as “slightly soluble”, “very slightly soluble”, “practically insoluble” and “insoluble” in USP 24, pp. 2254-2298; and compounds categorized as requiring 100 ml or more of water to dissolve 1 g of the drug, as listed in USP 24, pp. 2299-2304.

Illustratively, suitable drugs of low water solubility include, without limitation, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

Non-limiting illustrative examples of suitable drugs of low water solubility include acetohexamide, acetylsalicylic acid, alclofenac, allopurinol, atropine, benzthiazide, carprofen, celecoxib, chlordiazepoxide, chlorpromazine, clonidine, codeine, codeine phosphate, codeine sulfate, deracoxib, diacerein, diclofenac, diltiazem, eplerenone, estradiol, etodolac, etoposide, etoricoxib, fenbufen, fenclofenac, fenprofen, fentiazac, flurbiprofen, griseofulvin, haloperidol, ibuprofen, indomethacin, indoprofen, ketoprofen, lorazepam, medroxyprogesterone acetate, megestrol, methoxsalen, methylprednisone, morphine, morphine sulfate, naproxen, nicergoline, nifedipine, niflumic, oxaprozin, oxazepam, oxyphenbutazone, paclitaxel, phenindione, phenobarbital, piroxicam, pirprofen, prednisolone, prednisone, procaine, progesterone, pyrimethamine, rofecoxib, sulfadiazine, sulfamerazine, sulfisoxazole, sulindac, suprofen, temazepam, tiaprofenic acid, tilomisole, tolmetic, valdecoxib, etc.

The amount of drug incorporated in a drug-containing composition of the invention can be selected according to known principles of pharmacy. A therapeutically and/or prophylactically effective amount of drug is specifically contemplated, i.e., an amount of drug that is sufficient to elicit in a subject the required or desired therapeutic and/or prophylactic response when orally administered to the subject.

In a placebo suspension of the invention, the particulate substance of low water solubility is not a drug but an excipient. The particulate excipient of low water solubility can be inorganic or organic. Examples of suitable inorganic excipients of low water solubility include without limitation talc, silicon dioxide, titanium dioxide and zinc oxide. Examples of suitable organic excipients of low water solubility include without limitation stearic acid and microcrystalline cellulose.

A “water-soluble or swellable” polymer herein is a polymer that dissolves and/or swells in water. Examples of pharmaceutically acceptable water-soluble or swellable polymers include, without limitation, carbomer, carrageenan, gelatin, hydroxyethylcellulose, hydroxypropylcellulose, hydroxypropylmethylcellulose (HPMC), methylcellulose, povidone, polyethylene glycol (PEG) and sodium carboxymethylcellulose. Preferred polymers are gelatin, HPMC, povidone and PEG. An especially preferred polymer is povidone.

Povidone is also known as 1-ethenyl-2-pyrrolidinone homopolymer, 1-vinyl-2-pyrrolidinone polymer, poly[1-(2-oxo-1-pyrrolidinyl)ethylene], polyvidone, polyvinylpyrrolidone or PVP. It exists in different grades with varying molecular weight. The different grades are usually characterized by their K-value, which is calculated from the Fikentscher equation:

$$log z = c ( \frac{75 {k}^{2}}{1 + 1.5 kc} ) + k$$

where z is the relative viscosity of an aqueous solution with concentration c in % w/v of the polymer and k is the K-value ×10⁻³.

Preferably, the K-value of povidone useful in compositions of the invention is about 10 to about 120, more preferably about 30 to about 100, for example about 90. Average molecular weight of povidone useful in compositions of the invention is preferably about 2000 to about 3,000,000, more preferably about 400,000 to about 2,000,000, for example about 1,000,000.

PEG is also known as macrogol or polyoxyethylene glycol. Preferably, average molecular weight of PEG useful in compositions of the invention is about 190 to about 10,000.

A “nonsurfactant” polymer herein is a polymer such as those mentioned above that does not have significant surfactant properties and does not have strongly hydrophobic moieties. Where a surfactant polymer such as poloxamer (polyethylene-propylene glycol copolymer) is used in place of a nonsurfactant polymer in a composition of the invention, it is found that unacceptably high viscosity and/or phase separation can occur.

A particular advantage of a composition of the invention is that viscosity is low and substantially unaffected by the presence of the water-soluble or swellable nonsurfactant polymer. This makes the composition easier to dispense with accuracy and renders to the composition a palatable, non-slimy mouth feel. The lower viscosity is also an advantage during manufacturing of the composition, especially during mixing and filling into containers. Preferably the viscosity of the composition is less than about 5 Pa s (pascal second), more preferably less than about 2 Pa s, and most preferably less than about 1 Pa s, for example about 0.05 to about 1 Pa s. Viscosity herein refers to viscosity as measured by a rotation viscometer at low shear rate, e.g., a Haake CV100 at δ=2 s⁻¹ and 25° C.

It is believed, without being bound by theory, that an explanation for the enhanced mouth feel exhibited by compositions of the invention without substantial increase in viscosity is that the polymer is attracted to surfaces of the suspended drug or excipient particles, and can become adsorbed to these surfaces. Hence, it is believed that the particle surfaces are modified by the polymer to a degree effective to alter organoleptic properties of the suspension affected by direct contact between the suspended particles and oral mucosa, while the concentration of the polymer in the bulk of the aqueous carrier is low enough not to substantially increase viscosity of the suspension.

Adsorption of a polymer to suspended particles can be measured by several methods known to those skilled in the art. A presently preferred method to verify polymer adsorption to particles involves measuring free concentration of polymer, i.e., concentration of unadsorbed polymer, in the liquid carrier of a suspension following removal of suspended particles, for example by centrifugation and/or filtration, and comparing this free concentration with that of a corresponding polymer solution that has not been exposed to suspended particles. Adsorption of a polymer on suspended solid particles depends on the physical and chemical properties of the polymer and the solid including the structure and molecular weight of the polymer, the specific surface area of the solid particles, and affinity between polymer and particle surface, as well as the relative amounts of polymer and solid particles.

In one embodiment of the invention, the total amount of water-soluble or swellable nonsurfactant polymer is ineffective in at least one of the following respects: (a) to increase viscosity of the composition to a degree that significantly impairs pourability of the composition, and (b) to significantly increase rate of sedimentation or phase separation of the composition. Preferably the total amount of such polymer is ineffective in both these respects. In this context the term “increase” is to be understood to relate to a comparison of the composition of the invention with an otherwise similar composition lacking only the water-soluble or swellable nonsurfactant polymer, and prepared by a procedure similar in all respects except for addition of such polymer.

A significant impairment of pourability typically results when viscosity is increased more than about threefold and/or is increased to greater than about 5 Pa s. Preferably viscosity is not substantially increased, or is increased to not greater than about 2 Pa s. Most preferably viscosity is not substantially increased, or is increased to not greater than about 1 Pa s.

A significant increase in rate of sedimentation or phase separation herein is one that is readily observable within about 6 hours when the composition is allowed to stand without stirring or other agitation.

Typically a total amount of water-soluble or swellable nonsurfactant polymer ineffective to increase viscosity ofthe composition to a degree that significantly impairs pourability of the composition, or to significantly increase rate of sedimentation or phase separation ofthe composition, is an amount less than 1% by weight of the composition, for example an amount not greater than about 0.75% by weight of the composition.

In another embodiment, the total amount of such polymer is not greater than about 0.5% by weight of the composition. A minimum total amount of such polymer providing advantages according to the invention is about 0.01% by weight of the composition.

It has surprisingly been found that in some instances a total amount of such polymer of about 0.01% to about 0.2%, for example about 0.01% to about 0.1%, by weight of the composition significantly improves mouth feel in accordance with the invention.

A composition of the invention further comprises one or more suspending agents. Preferably an inorganic suspending agent, for example bentonite or magnesium aluminum silicate, is present, but organic suspending agents such as microcrystalline cellulose or xanthan gum can be used. A particularly preferred suspending agent is magnesium aluminum silicate.

Organoleptic properties of a composition of the invention can, if desired, be further enhanced by methods known in the art. In a drug-containing composition wherein the drug is one that does not have an unpleasant taste, e.g., because of extremely low solubility, the water-soluble or swellable nonsurfactant polymer alone can be sufficient to provide an organoleptically acceptable suspension.

In other cases, however, it can be desirable to further enhance organoleptic acceptability of a composition of the invention by addition of one or more sweetening and/or flavoring agents.

A “sweetening agent” herein is a substance that enhances sweetness of a composition, and includes soluble carbohydrates such as saccharose, glucose, fructose, mannitol and xylitol, and artificial sweeteners such as aspartame, neotame, acesulfame, cyclamic acid, saccharin and salts thereof.

A “flavoring agent” herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of suitable natural flavors, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, orange, peach, pear, peppermint, pineapple, raspberry, rose, spearmint, strawberry, tangerine, tea, vanilla, wintergreen, etc. Presently preferred flavoring agents include peppermint, vanilla, orange, grape, blackcurrant and pineapple. Also useful, especially in pediatric suspensions, is tutti-frutti or bubblegum flavor, a compounded flavoring agent based on fruit flavors.

Optionally, one or more taste modulators can also be present in a composition of the invention. Taste modulators are agents that affect a subject's perception of taste and include anesthetic agents.

Optionally, the solid particles can be taste-masked by mixing or coating the particles with one or more suitable taste-masking excipients as will be understood by a person skilled in the art of taste-masking.

If desired or necessary, one or more antimicrobial agents can be included in the composition. If the suspension is packed in a multidose container it is preferred that at least one pharmaceutically acceptable antimicrobial agent be present. Non-limiting illustrative examples of antimicrobial agents are benzalkonium chloride, benzoic acid and salts thereof (e.g., sodium benzoate), benzyl alcohol, butylparaben, cetrimide, chlorobutanol, editic acid, ethylparaben, glycerol, isopropyl alcohol, methylparaben, propylene glycol, propylparaben, sodium propionate and sorbic acid and salts thereof (e.g., potassium sorbate).

Preferred antimicrobial agents are methylparaben and sodium benzoate, for example in an amount of about 0.2% individually, or in a combination of about 0.2% in total, by weight of the composition.

Optionally, additional excipients can be added to improve other characteristics of the composition. Non-limiting examples of such additional excipients include, without limitation, surfactants, antioxidants, colorants and clays.

Although, as explained above, the advantages of the invention are especially great for a flocculated suspension, in one embodiment the composition of the invention is formulated as a substantially deflocculated and substantially physically stable suspension. This has the advantage of not needing any shaking or other agitation in order to redisperse drug particles prior to administration, although moderate shaking may still be recommended in order to make the suspension easier to pour if the suspension is thixotropic or shear-thinning.

Typically, given a flocculated suspension and a deflocculated suspension having the same drug particle size distribution, drug particle shape, drug particle density and carrier viscosity, drug particles in the deflocculated suspension sediment more slowly than those in the flocculated suspension. Additionally, when placed in a suspension carrier of low viscosity, for example an unstructured vehicle, deflocculated drug particles generally settle to form a tightly packed layer of sediment that is not readily dispersed upon moderate shaking. In contrast, flocculated particles generally settle to form a fluffy, loose layer of sediment that can be redispersed with moderate shaking.

An “unstructured vehicle” herein is a liquid vehicle that does not contain viscosity-imparting suspending agents in amounts that substantially reduce rate of sedimentation of dispersed particles.

The term “substantially physically stable” as used to describe a suspension herein means that (a) drug particles remain suspended in the suspension carrier such that dose uniformity is obtainable, as determined for example by volumetric means, during a stationary room temperature storage period of at least about 48 hours after the suspension is prepared, and/or (b) the suspension exhibits substantially uniform drug particle dispersion and substantially no phase separation during a stationary room temperature storage period of at least about 48 hours after preparation.

The term “dose uniformity” herein means that, with respect to two or more aliquots drawn volumetrically from the same suspension, either drawn simultaneously or at different time points and drawn from the same or different locations within the suspension, all aliquots contain substantially similar amounts (i.e., varying by no more than about 15%) of suspended drug and substantially similar amounts of dissolved drug. An amount of drug in a given volume of suspension can be measured by any suitable method, for example by high performance liquid chromatography (HPLC).

Suspensions ofthe invention can be prepared by any suitable process, not limited to processes described herein.

An illustrative process comprises (a) a step of dispersing one or more solid particulate substances of low water solubility in an aqueous liquid with agitation as needed to form a dispersion, (b) a step of adding to the dispersion one or more water-soluble or swellable nonsurfactant polymers with mixing as needed, to provide an intermediate suspension, and (c) a step of adding a suspending agent to the intermediate suspension with agitation as needed to form a homogeneous suspension of the invention. Optionally, one or more additional excipients can be added at any point or points in the process.

Another illustrative process comprises (a) a step of dissolving one or more water-soluble or swellable nonsurfactant polymers in an aqueous liquid to form a polymer solution, (b) a step of dispersing one or more drugs in the solution with agitation as needed to form a dispersion, and (c) a step of adding a suspending agent to the dispersion with agitation as needed to form a homogeneous suspension of the invention. Optionally, one or more additional excipients can be added at any point or points in the process.

It is important that the water-soluble or swellable nonsurfactant polymer be added before addition of the suspending agent.

A drug-containing suspension of the invention can be orally administered for diagnosis, treatment or prevention of any of a wide variety of diseases and adverse health conditions in a subject, depending on the drug present in the suspension. A “subject” herein to which the suspension can be administered includes a human subject of either sex and of any age, and also includes any nonhuman animal, particularly a warm-blooded animal, more particularly a domestic or companion animal, illustratively a cat, dog, cow or horse. In particular, a drug-containing suspension of the invention is therapeutically and/or prophylactically useful in diseases or health conditions that affect children or the elderly, and in diseases and health conditions that affect the subject's ability to swallow solid dosage forms such as tablets and capsules. The suspension is also particularly useful where it is desired or necessary to administer a drug in a large dose.

Where a suspension of the invention is to be used as a placebo, the drug can be replaced by a solid particulate excipient of low water solubility as illustrated herein. By appropriate selection of solid excipient and water-soluble or swellable nonsurfactant polymer and amounts thereof, a placebo suspension can be prepared that mimics organoleptic properties of a drug suspension, especially a flocculated drug suspension.

### EXAMPLES

The following examples illustrate aspects of the present invention but are not to be construed as limitations. Placebo suspensions are prepared using particles of talc and/or silicon dioxide (silica) instead of drug particles. Talc and silicon dioxide particles can be substituted by drug particles as will be understood by a person skilled in the art to prepare drug-containing suspensions of the invention having therapeutic and/or prophylactic utility.

### Example 1

A 7 kg batch of a suspension having the composition given in Table 1 was prepared.

TABLE 1
Composition of suspension of Example 1

| Ingredients | Amount (% w/w) |
|---|---|
| sorbitol solution 70% USP | 12.86 |
| fumaric acid (antioxidant) | 0.5 |
| methylparaben NF (antimicrobial) | 0.1 |
| colloidal silicon dioxide NF | 0.5 |
| talc USP, No. 141 | 1 |
| magnesium aluminum silicate NF, 11A | 2 |
| povidone K-90 | 0.1 |
| sodium chloride USP | 2 |
| sucrose NF, granular | 25 |
| peppermint flavor | 0.01 |
| vanilla flavor, A | 0.02 |
| pineapple flavor, A | 0.01 |
| orange flavor, N&A | 0.005 |
| FD&C Yellow #6 | 0.0028 |
| sodium hydroxide solution 40% w/v | to adjust pH to 2.9 |
| deionized water | q.s. ad 100 |

The preparation procedure was as follows:

1. To a stainless steel kettle, 3700 g of deionized water was added and heated to 40° C.

2. Sorbitol solution was added to the water in the kettle and the resulting solution was stirred for 3 minutes using a Sargent air-driven stirrer and a stainless steel propeller paddle with a diameter of 5.1 cm.

3. Fumaric acid and methylparaben were added to this solution and the resulting mixture stirred for 15 minutes.

4. Silicon dioxide was added to this mixture and the resulting dispersion was stirred for 5 minutes.

5. Talc was added to this dispersion and the resulting dispersion was stirred for 5 minutes.

6. Povidone was added to this dispersion with stirring for 10 minutes to form an intermediate suspension.

7. The heating of the kettle was stopped.

8. Magnesium aluminum silicate was added to the intermediate suspension and the resulting suspension was stirred for 10 minutes.

9. Sodium chloride was added to this suspension and the resulting suspension was stirred for 2 minutes.

10. Sucrose was added to this suspension and the resulting suspension was stirred for 2 minutes.

11. Flavors and coloring agents were added to this suspension and the resulting suspension was stirred for 5 minutes.

12. The pH of the suspension was adjusted to 2.9 (±0.5) with sodium hydroxide solution.

13. The suspension was weighed and deionized water was added to the suspension until the weight of the resulting suspension was 7.00 kg.

14. The suspension was transferred to a Koruma Mixer (DH-V 60/10, Germany).

15. The suspension was mixed for 5 minutes using Disho (homogenizer) at 5000 rpm and Scraper at speed 2 under vacuum (15 inches Hg).

16. The resulting finished suspension was then discharged.

### Example 2

Suspensions having compositions as described in Table 2 were prepared, substantially as described in Example 1.

TABLE 2
Composition of suspensions of Example 2

| | Amount (% w/w) | | | | | | | |
|---|---|---|---|---|---|---|---|---|
| Ingredient | Control | A1 | A2 | A3 | B1 | B2 | C1 | C2 |
| sorbitol solution 70% USP | 12.9 | 12.9 | 12.9 | 12.9 | 12.9 | 12.9 | 12.9 | 12.9 |
| fumaric acid | 0.5 | 0.5 | 0.5 | 0.5 | 0.5 | 0.5 | 0.5 | 0.5 |
| methylparaben | 0.1 | 0.1 | 0.1 | 0.1 | 0.1 | 0.1 | 0.1 | 0.1 |
| sodium benzoate NF | 0.1 | 0.1 | 0.1 | 0.1 | 0.1 | 0.1 | 0.1 | 0.1 |
| colloidal silicon dioxide NF | 0.5 | 0.5 | 0.5 | 0.5 | 0.5 | 0.5 | 0.5 | 0.5 |
| talc USP, No 141 | 1.0 | 1.0 | 1.0 | 1.0 | 1.0 | 1.0 | 1.0 | 1.0 |
| povidone K-90 | | 0.1 | 0.5 | 0.1* | | | | |
| polysorbate 80 (Tween 80) | | | | | 0.1 | 0.5 | | |
| poloxamer (Pluronic F-127) | | | | | | | 0.1 | 0.5 |
| magnesium aluminum silicate | 2.0 | 2.0 | 2.0 | 2.0 | 2.0 | 2.0 | 2.0 | 2.0 |
| sodium chloride USP | 2.0 | 2.0 | 2.0 | 2.0 | 2.0 | 2.0 | 2.0 | 2.0 |
| sucrose NF, granular | 25.0 | 25.0 | 25.0 | 25.0 | 25.0 | 25.0 | 25.0 | 25.0 |

| | |
|---|---|
| deionized water | g.s. ad 100 |

*In making this suspension, povidone was added after magnesium aluminum silicate.

The control suspension, which contains no povidone, was prepared for comparative purposes. Two preparations of the control suspension were made, one with and one without homogenizing.

Viscosity of the above suspensions was measured using a Haake CV100 measuring system consisting of RC20 Rheocontroller, RV20 Rotovisco, LV100 air supply unit, Haake F3 water bath and a computer with Rotation Version 2.1 software. Sample size was 1 gram with a P45 Cup and PK30-4 spindle. Viscosity was determined at δ=2 s⁻¹. Results are shown in Table 3.

TABLE 3
Viscosity of suspensions of Example 2

| Suspension | Viscosity (Pa s) |
|---|---|
| Control, without homogenizing | 0.098 |
| Control, with homogenizing | 0.290 |
| A1 | 0.605 |
| A2 | 0.097 |
| A3 | 1.764 |
| B1 | 1.360 |
| B2 | 0.538 |
| C1 | 2.917 |
| C2 | 0.818 |

The viscosity of suspensions containing 0.1 % or 0.5% povidone was low (about 0.1 to about 0.6 Pa s) except for suspension A3 where the povidone was added after the magnesium aluminum silicate. Replacement of povidone by polysorbate 80 or by poloxamer resulted in higher viscosity.

Suspensions A1, A3, B1 and C1 did not exhibit significantly greater phase separation or sedimentation than the control suspensions when allowed to stand in a test vial for 6 hours. However, of these suspensions, only suspension A1 had a viscosity of less than about 1 Pa s, as shown in Table 3 above. Suspension A2 exhibited slightly increased sedimentation than the control suspensions. Suspensions B2 and C2 exhibited severe and unacceptable phase separation.

The suspensions of Example 2 were submitted to a taste test. The control suspensions were reported to have an unpleasant sandy or powdery mouth feel. The mouth feel was significantly improved for suspensions A1 to A3 containing povidone.

It was concluded that of the suspensions tested, suspension A1 of the invention exhibited the most advantageous combination of improved mouth feel, low viscosity and resistance to phase separation and sedimentation. Suspension A2 of the invention exhibited increased sedimentation but was otherwise acceptable.

### Example 3

Suspensions having the composition listed in Table 4, with different amounts of methylparaben and/or sodium benzoate as antimicrobial agents, were prepared substantially as described in Example 1.

TABLE 4
Composition of suspensions of Example 3

| Ingredients | Amount (% w/w) |
|---|---|
| sorbitol solution 70% USP | 12.86 |
| fumaric acid | 0.5 |
| methylparaben and/or sodium benzoate | 0.1-0.2 (see Table 5) |
| colloidal silicon dioxide NF | 0.5 |
| talc USP, No 141 | 1 |
| magnesium aluminum silicate NF, 11A | 2 |
| povidone K-90 | 0.1 |
| sodium chloride USP | 2 |
| sucrose NF, granular | 25 |
| sodium hydroxide solution, 40% w/v | to adjust pH to 2.9 |
| deionized water | q.s. ad 100 |

These suspensions were subjected to antimicrobial effectiveness testing (AET) in accordance with USP 24, p. 1809. In addition to the species required by USP 24, namely Staphylococcus aureus (ATCC No. 6538), Pseudomonas aeruginosa (ATCC No. 9027), Escherichia coli (ATCC No. 8739), Candida albicans (ATCC No. 10231) and Aspergillus niger (ATCC No. 16404), the following supplemental isolates were used: Pseudomonas sp., Pseudomonas cepacia, Corynebacterium sp., Zygosaccharomyces rouxii and Penicillium sp. The AET results are shown in Table 5.

TABLE 5
AET results for suspensions of Example 3

| Suspension | Methylparaben | Sodium benzoate | AET |
|---|---|---|---|
| Ex. 3-1 | 0.1 | none | not effective |
| Ex. 3-2 | 0.2 | none | effective |
| Ex. 3-3 | none | 0.2 | effective |
| Ex. 3-4 | 0.1 | 0.1 | effective |

As shown in Table 5, presence of 0.2% by weight of methylparaben or sodium benzoate or a combination of 0.1 % by weight of methylparaben and 0.1 % by weight of sodium benzoate was sufficient to allow a suspension to pass the AET.

### Example 4

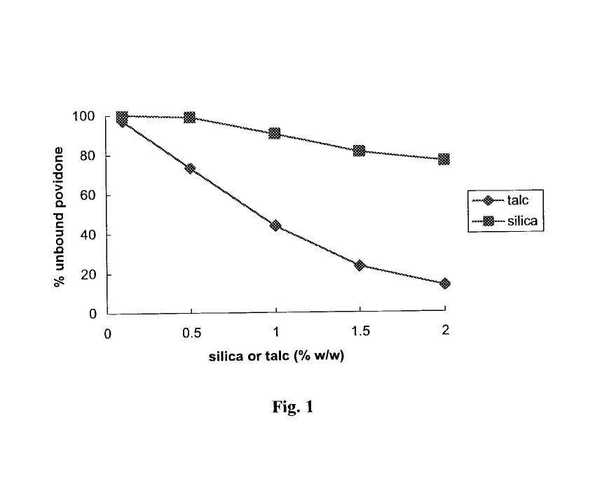

Ten samples were prepared by adding 0.1%, 0.5%, 1%, 1.5% and 2% by weight of colloidal silicon dioxide or talc to 0.1% aqueous povidone K-90 solution. These samples were homogenized at 13,000 rpm for 5 minutes. The samples were then left for 48 hours. Aliquots of all samples were filtered through a 0.22 μm filter. The amount of povidone in the filtrate was analyzed chromatographically. By comparing the area of the povidone peak for each sample with that for the initial aqueous povidone solution, the percentage of unbound povidone was obtained. Percentage of unbound povidone is plotted versus percentage of silicon dioxide or talc in FIG. 1. The decrease in unbound povidone with increasing concentration of silicon dioxide and talc suggests that the povidone is adsorbed onto the surfaces of solid particles.

What is claimed is:

1. A pharmaceutical composition suitable for oral administration, the composition comprising an aqueous medium having suspended therein a solid substance of low water solubility in particulate form, and further comprising a suspending agent and at least one pharmaceutically acceptable water-soluble or swellable nonsurfactant polymer, the total amount of all such polymers present being less than 1% by weight of the composition.

2. The composition of claim 1 wherein the total amount of all pharmaceutically acceptable water-soluble or swellable nonsurfactant polymers present is not greater than about 0.5% by weight of the composition.

3. The composition of claim 1 wherein the solid substance of low water solubility is a drug in a therapeutically and/or prophylactically effective amount.

4. The composition of claim 1 wherein the solid substance of low water solubility is an excipient and the composition is a placebo suspension.

5. The composition of claim 4 wherein said excipient is selected from the group consisting of talc, silicon dioxide, titanium dioxide and zinc oxide.

6. The composition of claim 1 having a viscosity at low shear rate of less than about 5 Pa s.

7. The composition of claim 1 having a viscosity at low shear rate of less than about 1 Pa s.

8. The composition of claim 1 that is a flocculated suspension.

9. The composition of claim 1 wherein the at least one water-soluble or swellable nonsurfactant polymer is selected from the group consisting of carbomer, carrageenan, gelatin, hydroxyethylcellulose, hydroxypropylcellulose, HPMC, methylcellulose, povidone, polyethylene glycol and sodium carboxymethylcellulose.

10. The composition of claim 1 wherein the at least one water-soluble or swellable nonsurfactant polymer is selected from the group consisting of gelatin, HPMC, povidone and polyethylene glycol.

11. The composition of claim 1 wherein the at least one water-soluble or swellable nonsurfactant polymer is povidone.

12. The composition of claim 11 wherein the povidone has a K-value of about 10 to about 120.

13. The composition of claim 11 wherein the povidone has a K-value of about 30 to about 100.

14. The composition of claim 1 further comprising one or more sweetening and/or flavoring agents.

15. The composition of claim 1 further comprising one or more antimicrobials.

16. A process for preparing a pharmaceutical composition, the process comprising a step of dispersing a solid particulate substance of low water solubility in an aqueous liquid to form a dispersion, a step of adding at least one water-soluble or swellable nonsurfactant polymer to the aqueous liquid, and a step of adding a suspending agent to the dispersion with mixing to form a suspension, wherein the suspending agent is added after addition of the polymer, and wherein the total amount of such polymer added is less than 1% by weight of the composition.

17. The process of claim 16 wherein the total amount of water-soluble or swellable nonsurfactant polymer added is not greater than about 0.5% by weight of the composition.

18. A method of diagnosis, treatment or prevention of a disease or other adverse health condition in a subject, the method comprising orally administering a composition of claim 3 to the subject.

19. The method of claim 18 wherein the subject is a human.

20. A method of conducting a clinical trial of a suspension formulation of a drug, the method comprising use of a composition of claim 4 as a comparator.
